SEQUENCE LISTING (4 sequences)

SEQ ID NO: 1 - DNA, 69 bp, synthetic construct
  source 1..69: mol_type = other DNA
gatcccgtttagagtgagcagaggcaacttcaagagagttgcctctgctcactctaaattttttccaaa

SEQ ID NO: 2 - DNA, 69 bp, synthetic construct
  source 1..69: mol_type = other DNA
agcttttggaaaaaatttagagtgagcagaggcaactctcttgaagttgcctctgctcactctaaacgg

SEQ ID NO: 3 - DNA, 69 bp, synthetic construct
  source 1..69: mol_type = other DNA
agtccggtcagaaaccagatggcgtttgttaaagaaacaaacgccatctggtatctgattttttccaaa

SEQ ID NO: 4 - DNA, 69 bp, synthetic construct
  source 1..69: mol_type = other DNA
agcttttggaaaaaatcagaaaccagatggcgtttgtctcttgaacaaacgccatctggtttctgacgg

(19) United States
(12) Patent Application Publication
(10) Pub. No.: US 2024/0076679 A1
(43) Pub. Date: Mar. 7, 2024

(54) GENE SILENCING THERAPY OF ACUTE RESPIRATORY DISORDER

(71) Applicant: Therapeutic Solutions International, Inc., Oceanside, CA (US)

(72) Inventors: Thomas E. ICHIM, Oceanside, CA (US); Timothy G. DIXON, Oceanside, CA (US); James VELTMEYER, Oceanside, CA (US)

(73) Assignee: Therapeutic Solutions International, Inc., Oceanside, CA (US)

(21) Appl. No.: 18/457,605

(22) Filed: Aug. 29, 2023

Related U.S. Application Data
(60) Provisional application No. 63/402,304, filed on Aug. 30, 2022.

(51) Int. Cl. C12N 15/113; A61P 37/06

(52) U.S. Cl. CPC C12N 15/1138; A61P 37/06; C12N 2310/14; C12N 2310/531

(57) ABSTRACT

Disclosed are treatment means, compositions of matter and protocols useful for suppression of acute respiratory disorder (ARDS) through induction of RNA interference in the pulmonary microenvironment alone and/or in conjunction with mucolytic and/or DNA disrupting agents. In one embodiment short interfering RNA (siRNA) is prepared which targets complement receptors C3R and/or C5R together with TNF-receptor, IL-6 receptor and/or TLR4 and TLR9. In some embodiments nanostilbene is utilized as a delivery vehicle for siRNA delivery.

### CROSS-REFERENCE TO RELATED APPLICATIONS

This application claims priority back to U.S. Provisional Application No. 63/402,304, titled “Gene Silencing Therapy of Acute Respiratory Disorder”, filed on Aug. 30, 2022, which is hereby incorporated by reference in its entirety.

### SEQUENCE LISTING

The application contains a Sequence Listing which has been submitted electronically in .XML format and is hereby incorporated by reference in its entirety. Said .XML copy, created on Oct. 20, 2023, is named “TSI_GSTARD_NP1_SL.xml” and is 4,775 bytes in size. The sequence listing contained in this .XML file is part of the specification and is hereby incorporated by reference herein in its entirety.

### FIELD OF THE INVENTION

The invention pertains to the field of pulmonary injury, more specifically, the invention pertains to the field of treatment of acute lung injury, more specifically the invention pertains to treatment of acute lung injury through selective gene silencing of inflammatory mediators.

### BACKGROUND OF THE INVENTION

Acute respiratory distress syndrome (ARDS) is a life-threatening condition. It is usually a complication of a serious existing condition, such as pneumonia, septicaemia, severe flu, or major trauma. ARDS has a substantial mortality rate and currently no effective treatments exist. ARDS is caused by an acute inflammatory response in the lungs which leads to hypoxia. It is believed that hypoxic conditions alter the function and survival of neutrophils resulting in a hyperinflammatory response that is damaging. In ARDS, there is an accumulation of dysfunctional neutrophils in the lungs. ARDS is associated with fluid accumulation in the lungs that is not explained by heart failure (noncardiogenic pulmonary edema), and is typically provoked by an acute injury to the lungs. This results in flooding of the lungs' microscopic air sacs, partial collapse of the lungs, and low levels of oxygen in the blood (hypoxemia). ARDS is associated with pathological findings including pneumonia, eosinophilic pneumonia, cryptogenic organizing pneumonia, acute fibrinous organizing pneumonia, and diffuse alveolar damage (DAD, which is characterized by a diffuse inflammation of lung tissue). The triggering insult to the tissue is often inflammation or mechanical stress in the lung, causing an initial release of chemical signals and other inflammatory mediators secreted by local epithelial and endothelial cells. Neutrophils and T-lymphocytes migrate into the inflamed lung tissue and amplify the syndrome. ARDS impairs the lungs' ability to exchange oxygen and carbon dioxide with the blood across a thin layer of the alveoli. A subject with ARDS presents diffuse injury to cells forming the barrier of the microscopic air sacs of the lungs, surfactant dysfunction, activation of the innate immune system response, and dysfunction of the body's regulation of clotting and bleeding. Signs and symptoms of ARDS can include shortness of breath, fast breathing, and a low oxygen level in the blood due to abnormal ventilation. ARDS has a death rate between 20 and 50%. Diagnostic criteria for ARDS were updated as the “Berlin definition” in 2012. Under that definition, ARDS is characterized by: lung injury of acute onset, within 1 week of an apparent clinical insult and with progression of respiratory symptoms; bilateral opacities on chest imaging not explained by other lung pathology; respiratory failure not explained by heart failure or volume overload; and decreased PaO.sub.2/FiO.sub.2 ratio (reduced arterial oxygenation from available inhaled gas). ARDS is usually treated with mechanical ventilation in the intensive care unit, but treatment of the underlying cause is crucial. If infection of the lugs is suspected, the patient must be aggressively treated with antibiotics as soon as possible. However, no actual treatment of the syndrome itself has been proven effective so far. There is thus a need in the art to identify novel therapeutic treatments that can be used to treat or prevent ARDS in an afflicted subject. The present invention addresses and meets this need.

### SUMMARY

Preferred embodiments include methods of treating acute respiratory disorder (ARDS) comprising the steps of: a) obtaining a patient suffering from ARDS; b) administering said patient a composition capable of inducing the process of RNA interference to one or more inflammatory mediators; and c) optionally providing a regenerative cell population to prevent long term effects of said ARDS.

Preferred methods include embodiments wherein said ARDS is induced by viral, bacterial, or non-infectious causes.

Preferred methods include embodiments wherein said ARDS is associated with a more than 35% increase in circulating C Reactive Protein.

Preferred methods include embodiments wherein said ARDS is associated with a more than 45% increase in interleukin-6.

Preferred methods include embodiments wherein said ARDS is associated with a more than 45% increase in interleukin-8.

Preferred methods include embodiments wherein said ARDS is associated with a more than 45% increase in interleukin-9.

Preferred methods include embodiments wherein said ARDS is associated with a more than 45% increase in interleukin-12.

Preferred methods include embodiments wherein said ARDS is associated with a more than 45% increase in interleukin-15.

Preferred methods include embodiments wherein said ARDS is associated with a more than 45% increase in interleukin-18.

Preferred methods include embodiments wherein said ARDS is associated with a more than 45% increase in interleukin-22.

Preferred methods include embodiments wherein said ARDS is associated with a more than 25% increase in interleukin-27.

Preferred methods include embodiments wherein said ARDS is associated with a more than 45% increase in interleukin-33.

Preferred methods include embodiments wherein said ARDS is associated with a more than 25% increase in interleukin-35.

Preferred methods include embodiments wherein said ARDS is associated with a more than 50% increase in pulmonary neutrophil count as determined by lavage.

Preferred methods include embodiments wherein said ARDS is associated with a more than 75% increase in pulmonary natural kill cell count as determined by lavage.

Preferred methods include embodiments wherein said ARDS is associated with a more than 10% increase in lung water content.

Preferred methods include embodiments wherein said ARDS is associated with a clinically significant decrease in respiratory capacity.

Preferred methods include embodiments wherein said composition capable of inducing the process of RNA interference is a double stranded RNA molecule.

Preferred methods include embodiments wherein said double stranded RNA molecule comprises more than 19 base pairs.

Preferred methods include embodiments wherein said double stranded RNA molecule comprises more than 15-100 base pairs.

Preferred methods include embodiments wherein said composition capable of inducing RNA interference is short hairpin RNA.

Preferred methods include embodiments wherein said RNA interference is used to downregulate expression of complement 5 receptor in pulmonary tissue.

Preferred methods include embodiments wherein said RNA interference is used to downregulate expression of complement 3 receptor in pulmonary tissue.

Preferred methods include embodiments wherein said RNA interference is used to downregulate expression of TNF p-55 receptor in pulmonary tissue.

Preferred methods include embodiments wherein said RNA interference is used to downregulate expression of TNF p-75 receptor in pulmonary tissue.

Preferred methods include embodiments wherein said RNA interference is used to downregulate expression of interferon alpha receptor in pulmonary tissue.

Preferred methods include embodiments wherein said RNA interference is used to downregulate expression of RIG1 in pulmonary tissue.

Preferred methods include embodiments wherein said RNA interference is used to downregulate expression of I-kappa B.

Preferred methods include embodiments wherein said RNA interference is used to downregulate expression of NF-kappa b in pulmonary tissue.

Preferred methods include embodiments wherein said RNA interference is used to downregulate expression of MDA-5 in pulmonary tissue.

Preferred methods include embodiments wherein said RNA interference is used to downregulate expression of ICAM=1 in pulmonary tissue.

Preferred methods include embodiments wherein said RNA interference is used to downregulate expression of interferon beta receptor in pulmonary tissue.

Preferred methods include embodiments wherein said RNA interference is used to downregulate expression of LFA-1 in pulmonary tissue.

Preferred methods include embodiments wherein said RNA interference is used to downregulate expression of ECAM in pulmonary tissue.

Preferred methods include embodiments wherein said RNA interference is used to downregulate expression of PECAM in pulmonary tissue.

Preferred methods include embodiments wherein said RNA interference is used to downregulate expression of CXCR4 in pulmonary tissue.

Preferred methods include embodiments wherein said RNA interference is used to downregulate expression of CCR7 in pulmonary tissue.

Preferred methods include embodiments wherein said RNA interference is used to downregulate expression of MIP-1 alpha in pulmonary tissue.

Preferred methods include embodiments wherein said RNA interference is used to downregulate expression of MIP-1 beta in pulmonary tissue.

Preferred methods include embodiments wherein said RNA interference is used to downregulate expression of RANTES in pulmonary tissue.

Preferred methods include embodiments wherein said RNA interference is used to downregulate expression of LIGHT in pulmonary tissue.

Preferred methods include embodiments wherein said regenerative cell is plastic adherent.

Preferred methods include embodiments wherein said plastic adherent cell is a mesenchymal stem cell.

Preferred methods include embodiments wherein said mesenchymal stem cells are derived from fluids.

Preferred methods include embodiments wherein said fluid is plasma.

Preferred methods include embodiments wherein said fluid is cerebral spinal fluid.

Preferred methods include embodiments wherein said fluid is urine.

Preferred methods include embodiments wherein said fluid is seminal fluid.

Preferred methods include embodiments wherein said mesenchymal stem cells are derived from tissues.

Preferred methods include embodiments wherein said tissue derived mesenchymal stem cells are selected from a group comprising of: a) bone marrow; b) perivascular tissue; c) adipose tissue; d) placental tissue; e) amniotic membrane; f) omentum; g) tooth; h) umbilical cord tissue; i) fallopian tube tissue; j) hepatic tissue; k) renal tissue; l) cardiac tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; v) skeletal muscle tissue; and w) subepithelial umbilical cord tissue.

Preferred methods include embodiments wherein said tissue derived mesenchymal stem cells are isolated from tissues containing cells selected from a group of cells comprising of: endothelial cells, epithelial cells, dermal cells, endodermal cells, mesodermal cells, fibroblasts, osteocytes, chondrocytes, natural killer cells, dendritic cells, hepatic cells, pancreatic cells, stromal cells, and salivary gland mucous cells.

Preferred methods include embodiments wherein said mesenchymal stem cells are plastic adherent.

Preferred methods include embodiments wherein said mesenchymal stem cells express a marker selected from a group comprising of: a) CD73; b) CD90; and c) CD105.

Preferred methods include embodiments wherein said mesenchymal stem cells are derived from umbilical cord tissue and lack expression of a marker selected from a group comprising of: a) CD14; b) CD45; and c) CD34.

Preferred methods include embodiments wherein said mesenchymal stem cells from umbilical cord tissue express markers selected from a group comprising of; a) oxidized low density lipoprotein receptor 1, b) chemokine receptor ligand 3; and c) granulocyte chemotactic protein.

Preferred methods include embodiments wherein said mesenchymal stem cells from umbilical cord tissue do not express markers selected from a group comprising of: a) CD117; b) CD31; c) CD34; and CD45;

Preferred methods include embodiments wherein said mesenchymal stem cells from umbilical cord tissue express, relative to a human fibroblast, increased levels of interleukin 8 and reticulon 1

Preferred methods include embodiments wherein said mesenchymal stem cells from umbilical cord tissue have the potential to differentiate into cells of at least a skeletal muscle, vascular smooth muscle, pericyte or vascular endothelium phenotype.

Preferred methods include embodiments wherein said mesenchymal stem cells from umbilical cord tissue express markers selected from a group comprising of: a) CD10; b) CD13; c) CD44; d) CD73; and e) CD90.

Preferred methods include embodiments wherein said umbilical cord tissue mesenchymal stem cell is an isolated umbilical cord tissue cell isolated from umbilical cord tissue substantially free of blood that is capable of self-renewal and expansion in culture,

Preferred methods include embodiments wherein said umbilical cord tissue mesenchymal stem cells has the potential to differentiate into cells of other phenotypes.

Preferred methods include embodiments wherein said other phenotypes comprise: a) osteocytic; b) adipogenic; and c) chondrogenic differentiation.

Preferred methods include embodiments wherein said cord tissue derived mesenchymal stem cells can undergo at least 20 doublings in culture.

Preferred methods include embodiments wherein said cord tissue derived mesenchymal stem cell maintains a normal karyotype upon passaging

Preferred methods include embodiments wherein said cord tissue derived mesenchymal stem cell expresses a marker selected from a group of markers comprised of: a) CD10 b) CD13; c) CD44; d) CD73; e) CD90; f) PDGFr-alpha; g) PD-L2; and h) HLA-A,B,C

Preferred methods include embodiments wherein said cord tissue mesenchymal stem cells does not express one or more markers selected from a group comprising of; a) CD31; b) CD34; c) CD45; d) CD80; e) CD86; f) CD117; g) CD141; h) CD178; i) B7-H2; j) HLA-G and k) HLA-DR,DP,DQ.

Preferred methods include embodiments wherein said umbilical cord tissue-derived cell secretes factors selected from a group comprising of: a) MCP-1; b) MIP1beta; c) IL-6; d) IL-8; e) GCP-2; f) HGF; g) KGF; h) FGF; i) HB-EGF; j) BDNF; k) TPO; 1) RANTES; and m) TIMP1

Preferred methods include embodiments wherein said umbilical cord tissue derived cells express markers selected from a group comprising of: a) TRA1-60; b) TRA1-81; c) SSEA3; d) SSEA4; and e) NANOG.

Preferred methods include embodiments wherein said umbilical cord tissue-derived cells are positive for alkaline phosphatase staining.

### DETAILED DESCRIPTION OF THE INVENTION

The invention provides the use of RNA interference inducing compositions in order to evoke gene silencing in a patient suffering from or predisposed to ARDS. In a preferred embodiment said gene silencing is accomplished by use of short interfering RNA. Pulmonary targets for said RNA interference inducing molecules include: TNF-alpha receptors (p55/p75), interferon receptors, interleukins, chemokines, inflammatory transcription factors and complement receptors.

The invention discloses the application of gene silencing technology for the treatment of ARDS. It has been reported that RNAi (RNA interference) is a phenomenon capable of inducing the selective degradation of target gene mRNA so as to silence the target gene expression by introducing into cells a double-stranded RNA that comprises a sense RNA having the sequence homologous to the target gene mRNA and an antisense RNA having the sequence complementary to the sense RNA. RNAi, because of its capability to selectively silence the target gene expression, has received considerable attention as a simple gene knock-down method that replaces the conventional gene disruption method relying on the tedious, inefficient homologous recombination. Studies have reported that RNAi can be induced also in mammalian cells by transducing the cells with short dsRNAs of 21 or 22 nucleotide long having a single-stranded 2 or 3 nucleotide of 3′-overhang terminus in place of long dsRNAs as those used in other organisms. In order to evoke the process of RNAi, an RNAi-inducing entity, siRNA (small interference RNA) is a short, double-helix RNA strand consisting of about 19 to 23 nucleotides, which can suppress expression of a targeted mRNA being related to a disease and having complementary base sequence to the siRNA. However, since siRNA has very low stability and is quickly degraded in vivo, its therapeutic efficiency deteriorates quickly. Even though the dose of expensive siRNA can be increased, the anionic nature of siRNA hinders it from permeating a cell membrane with negative charge, leading to low levels of siRNA transfer into intracellular compartments (Chemical and Engineering News Dec. 22, 32-36, 2003). In addition, the linkage of a ribose sugar in siRNA is chemically very unstable, and thus the majority of siRNA has a half-life of about 30 minutes in vivo and is quickly degraded.

As used in this disclosure, except where the context requires otherwise, the term “comprise” and variations of the term, such as “comprising,” “comprises” and “comprised” are not intended to exclude other additives, components, integers or steps.

As used in this disclosure, the term “substantially complementary” and variations of the term, such as “substantial complement,” means that at least 90% of all of the consecutive residues in a first strand are complementary to a series of consecutive residues of the same length of a second strand. As will be understood by those with skill in the art with reference to this disclosure, one strand can be shorter than the other strand and still be substantially complementary. With respect to the invention disclosed in this disclosure, for example, the RNAi, siRNA or shRNA can be shorter or longer than the complementary messenger RNA (mRNA) for the target gene interest; however, it is preferable that the RNAi molecule is shorter than and substantially complementary to its corresponding mRNA.

As used in this disclosure “RNAi molecule” refers to a nucleic acid that forms a double stranded RNA, which double stranded RNA has the ability to reduce or inhibit expression of a gene or target gene when the RNAi molecule present in the same cell as the gene or target gene. In general, RNAi molecules are fragments of double-stranded RNA (dsRNA), which share a homologous sequence with a target gene. The dsRNA of an RNAi molecule typically contains a “sense” sequence corresponding a partial sequence of the target gene messenger RNA (mRNA) and an “antisense” sequence that is substantially complementary and capable of specifically hybridizing to a target gene mRNA.

By “small interfering RNA” (siRNA) is meant an RNA molecule which down-regulates or silences (prevents) the expression of a gene/mRNA of its endogenous cellular counterpart. RNA interference (RNAi) refers to the process of sequence-specific post transcriptional gene silencing in mammals mediated by small interfering RNAs (siRNAs) (Fire et al, 1998, Nature 391, 806). The corresponding process in plants is commonly referred to as specific post transcriptional gene silencing or RNA silencing and is also referred to as quelling in fungi. The RNA interference response may feature an endonuclease complex containing an siRNA, commonly referred to as an RNA-induced silencing complex (RISC), which mediates cleavage of single-stranded RNA having sequence complementary to the antisense strand of the siRNA duplex. Cleavage of the target RNA may take place in the middle of the region complementary to the antisense strand of the siRNA duplex (Elbashir et al 2001, Genes Dev., 15, 188). For information on these terms and proposed mechanisms, see Bernstein E., et al., 2001 November; 7(11):1509-21; and Nishikura K.: Cell. 2001. 107(4):415-8. Examples of siRNA molecules which are used in the present application are provided in Tables A1-A18, B1-B15 and C1-C2.

RNAi molecules include small interfering RNAs (siRNAs), which are comprised of short dsRNA molecules. In one embodiment, a siRNA comprises a dsRNA containing an antisense sequence substantially or completely complementary to a target gene mRNA. The portions of the siRNA that hybridize to form the dsRNA are typically substantially or completely complementary to each other. The sequences of the siRNA can correspond to the full length target gene, or a subsequence thereof. Typically, the siRNA is at least about 15-50 nucleotides in length (e.g., each complementary sequence of the double stranded siRNA is 15-50 nucleotides in length, and the double stranded siRNA is about 15-50 base pairs in length), preferably about 19-27 base pairs in length, e.g., 19, 20, 21, 22, 23, 24, 25, 26 or 27 nucleotides in length.

In a preferred embodiment, the double stranded portion of the siRNA is about 19-23 base pairs and contains two-base single-stranded overhangs on each end, mimicking the product naturally produced by the endoribonuclease Dicer in vivo. Suitable siRNAs are integrated into a multiprotein complex called the RNA-induced silencing complex (RISC), which initiates the degradation of homologous mRNA.] Synthesis of the siRNA can readily be accomplished by phosphoramidite chemistry and can be obtained from a number of commercial sources well known in the art, as will be understood by those with skill in the art with reference to this disclosure. An alternative to individual chemical synthesis of siRNA is to construct a sequence for insertion in an expression vector. Several RNAi vectors for the transcription of inserts are commercially available (e.g., Ambion, Austin, Tex.; Invitrogen, Carlsbad, Calif.). Some use an RNA polymerase III (Pol III) promoter to drive expression of both the sense and antisense strands separately, which then hybridize in vivo to make the siRNA. Accordingly, RNAi molecules also include short “hairpin” RNA (shRNA), which functions in a similar manner as siRNA. Whereas siRNA is comprised of two strands of complementary RNA that can be synthesized, a shRNA is encoded by DNA as a single RNA molecule that hybridizes to itself with a loop at one end. The “hairpin” loop of the shRNA is cleaved intracellularly yielding a molecule similar to a siRNA. A typical shRNA vector design incorporates two inverted repeats, containing the sense and antisense target sequences, separated by a loop sequence. Commonly used loop sequences contain 8-9 bases. A terminator sequence consisting of 5-6 poly dTs may be present at the 3′ end and cloning sequences can be added to the 5′ ends of the complementary oligonucleotides

The present invention features “small interfering RNA molecules” (“siRNA molecules” or “siRNA”) and methods (e.g., therapeutic methods) for using said siRNA molecules. An siRNA molecule of the instant invention is preferably a duplex consisting of a sense strand and complementary antisense strand, the antisense strand having sufficient complementarity to a target mRNA to mediate RNAi. Because only the antisense strand of an siRNA duplex incorporates into the RISC to mediate cleavage or silencing of the target mRNA, a single antisense strand capable of activating RISC (e.g., a stable form of the antisense strand) could mimic the functionality of the siRNA duplex. Preferably, the strands of an siRNA duplex are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed. Preferably, the siRNA molecule has a length from about 10-50 or more nucleotides, i.e., each strand comprises 10-50 nucleotides (or nucleotide analogs). More preferably, the siRNA molecule has a length from about 15-45 or 15-30 nucleotides. Even more preferably, the siRNA molecule has a length from about 16-25 or 18-23 nucleotides. The siRNA molecules of the invention further have a sequence that is “sufficiently complementary” to a target mRNA sequence to direct target-specific RNA interference (RNAi), as defined herein, i.e., the siRNA has a sequence sufficient to trigger the destruction of the target mRNA by the RNAi machinery or process.

siRNAs function as the specificity determinants of the RNAi pathway, where they act as guides to direct endonucleolytic cleavage of their target RNAs. The two strands of an siRNA are not equally eligible for assembly into RISC. Rather, both the absolute and relative stabilities of the base pairs at the 5′ ends of the two siRNA strands determine the degree to which each strand participates in the RNAi pathway. siRNA duplexes can be functionally asymmetric, with only one of the two strands able to trigger RNAi. Asymmetry is also the hallmark of a related class of small, single-stranded, non-coding RNAs, microRNAs (miRNAs). In general, siRNA containing nucleotide sequences sufficiently identical to a portion of the target gene to effect RISC-mediated cleavage of the target gene are preferred. 100% sequence identity between the siRNA and the target gene is not required to practice the present invention. The invention can tolerate sequence variations within the methods, tissues and compositions of the invention in order to enhance efficiency and specificity of RNAi. For example, siRNA sequences with insertions, deletions, and single point mutations relative to the target sequence can also be effective for inhibition. Alternatively, siRNA sequences with nucleotide analog substitutions or insertions can be effective for inhibition. The siRNAs of the invention can comprise 16-30, e.g., 16, 17, 18, 19, 20, 21, 22, 23, 24, 25, 26, 27, 28, 29, or 30 nucleotides in each strand, wherein one of the strands is substantially complementary, e.g., at least 80% complementary (or more, e.g., 85%, 90%, 95%, or 100%)(for example, having 3, 2, 1, or 0 mismatched nucleotide(s)), to a target region. A target region differs by at least one base pair between the wild type and mutant allele, e.g., a target region comprising a gain-of-function mutation, and the other strand is identical or substantially identical to the first strand. The dsRNA molecules of the invention can be chemically synthesized or can be transcribed be in vitro from a DNA template or engineered RNA precursor. The dsRNA molecules can be designed using any method known in the art, for instance, by using the following protocol:

1. Beginning with an AUG start codon, search for AA dinucleotide sequences; each AA and the 3′ adjacent 16 or more nucleotides are potential siRNA targets. The siRNA should be specific for a target region that differs by at least one base pair between the wild type and mutant allele, e.g., a target region comprising the gain-of-function mutation. In cases where the gain-of-function mutation is associated with one or more other mutations in the same gene, the siRNA can be targeted to any of the mutations. In some cases, the siRNA is targeted to an allelic region that does not comprise a known mutation but does comprise an allelic variation of the wild-type (reference) sequence. The first strand should be complementary to this sequence, and the other strand is identical or substantially identical to the first strand. In one embodiment, the nucleic acid molecules are selected from a region of the target allele sequence beginning at least 50 to 100 nt downstream of the start codon, e.g., of the sequence of Insulin 2. Further, siRNAs with lower G/C content (35-55%) may be more active than those with G/C content higher than 55%. Thus in one embodiment, the invention includes nucleic acid molecules having 35-55% G/C content. In addition, the strands of the siRNA can be paired in such a way as to have a 3′ overhang of 1 to 4, e.g., 2, nucleotides. Thus in another embodiment, the nucleic acid molecules can have a 3′ overhang of 2 nucleotides, such as TT. The overhanging nucleotides can be either RNA or DNA.

2. Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at www.ncbi.nlm.nih.gov/BLAST.

3. Select one or more sequences that meet your criteria for evaluation. Further general information about the design and use of siRNA may be found in “The siRNA User Guide,” available at http://www.mpibpc.gwdg.de/abteilungen/100/105/sima.html. The siRNAs of the invention may have one or more modified bases in the antisense strand, e.g., U(5Br), U(51), and/or DAP. Such modified siRNAs can be synthesized with the modified base. Negative control siRNAs should have the same nucleotide composition as the selected siRNA, but without significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

Sequence identity may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology=# of identical positions/total # of positions.times.100), optionally penalizing the score for the number of gaps introduced and/or length of gaps introduced. The comparison of sequences and determination of percent identity between two sequences can be accomplished using a mathematical algorithm. For certain applications, the alignment can be generated over a certain portion of the sequence aligned having sufficient identity but not over portions having low degree of identity (i.e., a local alignment). The alignment may also be optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17):3389-3402. In another embodiment, the alignment is optimized by introducing appropriate gaps and percent identity is determined over the entire length of the sequences aligned (i.e., a global alignment).). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Greater than 80% sequence identity, e.g., 80%, 81%, 82%, 83%, 84%, 85%, 86%, 87%, 88%, 89%, 90%, 91%, 92%, 93%, 94%, 95%, 96%, 97%, 98%, 99% or even 100% sequence identity, between the siRNA antisense strand and the portion of the target gene is preferred. Alternatively, the siRNA may be defined functionally as a nucleotide sequence (or oligonucleotide sequence) that is capable of hybridizing with a portion of the target gene transcript (e.g., 400 mM NaCl, 40 mM PIPES pH 6.4, 1 mM EDTA, 50.degree. C. or 70.degree. C. hybridization for 12-16 hours; followed by washing). Additional preferred hybridization conditions include hybridization at 70.degree. C. in 1.times.SSC or 50.degree. C. in 1.times.SSC, 50% formamide followed by washing at 70.degree. C. in 0.3.times.SSC or hybridization at 70.degree. C. in 4.times.SSC or 50.degree. C. in 4.times.SSC, 50% formamide followed by washing at 67.degree. C. in 1.times.SSC. The hybridization temperature for hybrids anticipated to be less than 50 base pairs in length should be 5-10.degree. C. less than the melting temperature (Tm) of the hybrid, where Tm is determined according to the following equations. For hybrids less than 18 base pairs in length, Tm(.degree. C.)=2(# of A+T bases)+4(# of G+C bases). For hybrids between 18 and 49 base pairs in length, Tm(.degree. C.)=81.5+16.6(log 10[Na+])+0.41(% G+C)−(600/N), where N is the number of bases in the hybrid, and [Na+] is the concentration of sodium ions in the hybridization buffer ([Na+] for 1.times.SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference. The length of the identical nucleotide sequences may be at least about 10, 12, 15, 17, 20, 22, 25, 27, 30, 32, 35, 37, 40, 42, 45, 47 or 50 bases. The RNA molecules of the present invention can be modified to improve stability in serum or in medium for cell and/or organ cultures. In order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect the efficiency of RNA interference. In a preferred aspect, the invention features small interfering RNAs (siRNAs) that include a sense strand and an antisense strand, wherein the antisense strand has a sequence sufficiently complementary to a target mRNA sequence to direct target-specific RNA interference (RNAi) and wherein the sense strand and/or antisense strand is modified by the substitution of internal nucleotides with modified nucleotides, such that in vivo stability is enhanced as compared to a corresponding unmodified siRNA. RNA molecules of the invention may additionally contain at least one modified nucleotide analogue. The nucleotide analogues may be located at positions where the target-specific activity, e.g., the RNAi mediating activity is not substantially effected, e.g., in a region at the 5′-end and/or the 3′-end of the RNA molecule. Particularly, the ends may be stabilized by incorporating modified nucleotide analogues. Preferred nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In preferred backbone-modified ribonucleotides the phosphoester group connecting to adjacent ribonucleotides is replaced by a modified group, e.g., of phosphothioate group. In preferred sugar-modified ribonucleotides, the 2′ OH-group is replaced by a group selected from H, OR, R, halo, SH, SR, NH.sub.2, NHR, NR.sub.2 or ON, wherein R is C.sub.1-C.sub.6 alkyl, alkenyl or alkynyl and halo is F, Cl, Br or I. Exemplary art-recognized modifications of RNAi agents include, e.g., 2-Fluoro and 2-Chloro modifications and other stabilizing modifications, such as 2′-O-Me modifications and locked nucleic acids (LNA).

Also preferred are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined. Additional modified and conjugated forms of RNAi agents may also be used in the methods of the present invention, including, e.g., cholesterol conjugation, as such modification has been found to significantly improve in vivo pharmacological properties (Soutschek J, et al. Nature 2004 432: 173-78). RNA may be produced enzymatically or by partial/total organic synthesis, any modified nibonucleotide can be introduced by in vitro enzymatic or organic synthesis. In one embodiment, an RNAi agent is prepared chemically. Methods of synthesizing RNA molecules are known in the art, in particular, the chemical synthesis methods as de scribed in Verma and Eckstein (1998) Annul Rev. Biochem. 67:99-134. In another embodiment, an RNAi agent (e.g. a siRNA) is prepared enzymatically. For example, a ds-siRNA can be prepared by enzymatic processing of a long ds RNA having sufficient complementarity to the desired target mRNA. Processing of long ds RNA can be accomplished in vitro, for example, using appropriate cellular lysates and ds-siRNAs can be subsequently purified by gel electrophoresis or gel filtration. ds-siRNA can then be denatured according to art-recognized methodologies. In an exemplary embodiment, RNA can be purified from a mixture by extraction with a solvent or resin, precipitation, electrophoresis, chromatography, or a combination thereof. Alternatively, the RNA may be used with no or a minimum of purification to avoid losses due to sample processing. Alternatively, the siRNA can also be prepared by enzymatic transcription from synthetic DNA templates or from DNA plasmids isolated from recombinant bacteria. Typically, phage RNA polymerases are used such as T7, T3 or SP6 RNA polymerase (Milligan and Uhlenbeck (1989) Methods Enzymol. 180:51-62). The RNA may be dried for storage or dissolved in an aqueous solution. The solution may contain buffers or salts to inhibit annealing, and/or promote stabilization of the single strand.

The instant invention additionally provides for delivery of shRNAs having enhanced specificity or efficacy in mediating RNAi. In contrast to short siRNA duplexes, short hairpin RNAs (shRNAs) mimic the natural precursors of miRNAs and enter at the top of the RNAi pathway. For this reason, shRNAs are believed to mediate RNAi more efficiently by being fed through the entire natural RNAi pathway. shRNAs have a single strand that forms a duplex stem including two portions that are generally complementary, and a loop, that connects the two portions of the stem. In a preferred embodiment, short hairpin RNAs of the invention are artificial constructs engineered to deliver desired siRNAs. In shRNAs employed in certain embodiments of the instant invention, one portion of the duplex stem is a nucleic acid sequence that is complementary (or anti-sense) to the target mRNA. Thus, shRNAs include a duplex stem with two portions and a loop connecting the two stem portions. The two stem portions are about 18 or 19 to about 25, 30, 35, 37, 38, 39, or 40 or more nucleotides in length. When used in mammalian cells, the length of the stem portions should be less than about 30 nucleotides to avoid provoking non-specific responses like the interferon pathway. In non-mammalian cells, the stem can be longer than 30 nucleotides. In fact, the stem can include much larger sections complementary to the target mRNA (up to, and including the entire mRNA). The two portions of the duplex stem must be sufficiently complementary to hybridize to form the duplex stem. Thus, the two portions can be, but need not be, fully or perfectly complementary. In addition, the two stem portions can be the same length, or one portion can include an overhang of 1, 2, 3, or 4 micleotides. The overhanging nucleotides can include, for example, uracils (Us), e.g., all Us. The loop in the shRNAs can be 2, 3, 4, 5, 6, 7, 8, 9, or more, e.g., 15 or 20, or more nucleotides in length. shRNAs of the invention include the sequences of the desired siRNA duplex. The desired siRNA duplex, and thus both of the two stem portions in the shRNA, are selected by methods known in the art. The shRNAs of the invention can be synthesized by standard methods known in the art, e.g., by use of an automated DNA synthesizer (such as are commercially available from Biosearch, Applied Biosystems, etc.). The shRNAs can be used directly as described below or cloned into expression cassettes or vectors by methods known in the field. Such cassettes or vectors can be constructed by recombinant DNA technology methods known in the art. Vectors can be plasmid, viral, or other vectors known in the art such as those described herein, used for replication and expression in mammalian cells or other targeted cell types. The nucleic acid sequences encoding the shRNAs of the invention can be prepared using known techniques. For example, two synthetic DNA oligonucleotides can be synthesized to create a novel gene encoding the entire shRNA. The DNA oligonucleotides, which will pair, leaving appropriate ‘sticky ends’ for cloning, can be inserted into a restriction site in a plasmid that contains a promoter sequence (e.g., a Pol 11 or a Pol III promoter) and appropriate terminator sequences 3′ to the shRNA sequences (e.g., a cleavage and polyadenylation signal sequence from SV40 or a Pol III terminator sequence). The expression of the shRNAs of certain embodiments of the invention is driven by regulatory sequences, and the vectors of the invention can include any regulatory sequences known in the art to act in mammalian cells. The term regulatory sequence includes promoters, enhancers, and other expression control elements. A person skilled in the art would be able to choose the appropriate regulatory sequence.

In the practice of the invention, it is important to note that downregulation is the process by which a cell decreases the number of a cellular component, such as RNA or protein in response to external variable. RNAi down regulates a gene function by mRNA degradation. Thus, the degree of RNA interference achieved is directly proportional to the level of mature mRNA and the translated proteins. The terms “downregulate,” “downregulation,” “downregulating” or “downregulated” interchangeably refer to a protein or nucleic acid (RNA) that is transcribed or translated at a detectably lower level, in comparison to a normal or untreated cell. Downregulation can be detected using conventional techniques for detecting and/or measuring target mRNA (i.e., RT-PCR, PCR, hybridization) or target proteins (i.e., ELISA, immunohistochemical techniques, enzyme activity). Downregulation can be 10%, 20%, 30%, 40%, 50%, 60%, 70%, 80%, 90% etc. in comparison to a normal or untreated cell. In certain instances, downregulation is 1-fold, 2-fold, 3-fold, 4-fold or more lower levels of transcription or translation in comparison to a normal or untreated cell.

### Example 1: Treatment of Endotoxin Induced ARDS by siRNA Targeting Complement C5a Receptor

Mice were randomly divided into four groups with 4-5 animals per group. Mice were anesthetized with an i.p. injection of 1% pentobarbital sodium (50 mg/kg, Merck, Germany). After that, LPS was intratracheally delivered into the lungs of mice at doses of 10.0 mg/kg body weight to induce the ALI model.

As for siRNA-treated groups, 50 μg of C5aR siRNA plasmid DNA were diluted in 1 ml of PBS and injected into mice by tail vein by a “hydrodynamic” injection. Injections were performed 3 hours after LPS challenge.

Mice were divided into 3 groups, Group 1 Endotoxin Injection. Group 2 Endotoxic Injection and C5 siRNA. Group 3 Endotoxin and scrambled siRNA. Experiments were performed in 3 independent times. Survival was assessed on days 2 and 4.. The oligonucleotides containing sequences specific for C5 was

(SEQ ID NO: 1)
5′GATCCCGTTTAGAGTGAGCAGAGGCAACTTCAAGAGAGTTGCCTCTG
CTCACTCTAAATTTTTTCCAA A-3′
and
(SEQ ID NO: 2)
5′AGCTTTTGGAAAAAATTTAGAGTGAGCAGAGGCAACTCTCTTGAAGT
TGCCTCTGCTCACTCTAAACGG-3′

For control scrambled 3′; #2:

(SEQ ID NO: 3)
5′AGTCCGGTCAGAAACCAGATGGCGTTTGTTAAAGAAACAAACGCCAT
CTGGTATCTGATTTTTTCCAA A-3′
and
(SEQ ID NO: 4)
5′AGCTTTTGGAAAAAATCAGAAACCAGATGGCGTTTGTCTCTTGAACA
AACGCCATCTGGTT TCTGACG G-3′ were synthesized and
annealed.

A C5aR siRNA expression vector, which expresses hairpin siRNA under the control of the mouse U6 promoter and cGFP genes, was constructed by inserting pairs of annealed DNA oligonucleotides into a pRNAT-U6.1/Neo siRNA expression vector that had been digested with BamH I and HindIII (Genescript, Piscataway, NJ).

As seen below a significant survival advantage was bestowed upon mice receiving C5 specific siRNA.

| | Experiment 1 | | | Experiment 2 | | | Experiment 3 | | |
|---|---|---|---|---|---|---|---|---|---|
| | | Group 2 | Group 3 | | Group 2 | Group 3 | | Group 2 | Group 3 |
| | | Endo- | Endo- | | Endo- | Endo- | | Endo- | Endo- |
| | Group 1 | toxin + | toxin + | Group 1 | toxin + | toxin + | Group 1 | toxin + | toxin + |
| | Endo- | C5 | scramble | Endo- | C5 | scramble | Endo- | C5 | scramble |
| | toxin | siRNA | siRNA | toxin | siRNA | siRNA | toxin | siRNA | siRNA |
| Pre | 10 | 10 | 10 | 10 | 10 | 10 | 10 | 10 | 10 |
| Day 2 | 4 | 10 | 4 | 5 | 9 | 3 | 2 | 8 | 3 |
| Day 4 | 0 | 8 | 1 | 1 | 7 | 1 | 0 | 7 | 1 |

1. A method of treating acute respiratory disorder (ARDS) comprising the steps of: a) obtaining a patient suffering from ARDS; b) administering said patient a composition capable of inducing the process of RNA interference to one or more inflammatory mediators; and c) providing a regenerative cell population to prevent long term effects of said ARDS.

2. The method of claim 1, wherein said composition capable of inducing the process of RNA interference is a double stranded RNA molecule.

3. The method of claim 2, wherein said double stranded RNA molecule comprises more than 19 base pairs.

4. The method of claim 2, wherein said double stranded RNA molecule comprises more than 15-100 base pairs.

5. The method of claim 1, wherein said composition capable of inducing RNA interference is short hairpin RNA.

6. The method of claim 1, wherein said RNA interference is used to downregulate expression of complement component 5 in pulmonary tissue.

7. The method of claim 1, wherein said regenerative cell is plastic adherent.

8. The method of claim 7, wherein said plastic adherent cell is a mesenchymal stem cell.

9. The method of claim 8, wherein said mesenchymal stem cells are selected from the group consisting of: a) bone marrow; b) perivascular tissue; c) adipose tissue; d) placental tissue; e) amniotic membrane; f) omentum; g) tooth; h) umbilical cord tissue; i) fallopian tube tissue; j) hepatic tissue; k) renal tissue; l) cardiac tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; v) skeletal muscle tissue; and w) subepithelial umbilical cord tissue.

10. The method of claim 9, wherein said mesenchymal stem cells express a marker selected from the group consisting of: a) CD73; b) CD90; and c) CD105.

11. The method of claim 9, wherein said mesenchymal stem cells are derived from umbilical cord tissue and lack expression of a marker selected from the group consisting of: a) CD14; b) CD45; and c) CD34.

12. The method of claim 9, wherein said mesenchymal stem cells from umbilical cord tissue express markers selected from the group consisting of; a) oxidized low density lipoprotein receptor 1, b) chemokine receptor ligand 3; and c) granulocyte chemotactic protein.

13. The method of claim 9, wherein said mesenchymal stem cells from umbilical cord tissue do not express markers selected from the group consisting of: a) CD117; b) CD31; c) CD34; and CD45.

14. The method of claim 9, wherein said mesenchymal stem cells from umbilical cord tissue express, relative to a human fibroblast, increased levels of interleukin 8 and reticulon 1.

15. The method of claim 14, wherein said mesenchymal stem cells from umbilical cord tissue have the potential to differentiate into cells of at least a skeletal muscle, vascular smooth muscle, pericyte or vascular endothelium phenotype.

16. The method of claim 14, wherein said mesenchymal stem cells from umbilical cord tissue express markers selected from the group consisting of: a) CD10; b) CD13; c) CD44; d) CD73; and e) CD90.

17. The method of claim 14, wherein said umbilical cord tissue mesenchymal stem cell is an isolated umbilical cord tissue cell isolated from umbilical cord tissue substantially free of blood that is capable of self-renewal and expansion in culture.

18. The method of claim 17, wherein said other phenotypes comprise: a) osteocytic; b) adipogenic; and c) chondrogenic differentiation.

19. The method of claim 17, wherein said cord tissue derived mesenchymal stem cells can undergo at least 20 doublings in culture.

20. The method of claim 17, wherein said cord tissue derived mesenchymal stem cell maintains a normal karyotype upon passaging.
